(19) United States
(12) Patent Application Publication
(10) Pub. No.: US 2021/0162563 A1
(43) Pub. Date: Jun. 3, 2021

(54) Particle Blast System, and Blast Device and Recipient Therefor

(71) Applicant: GenR8 BVBA, Berchem-Antwerpen (BE)

(72) Inventors: Koen Meukens, Berchem-Antwerpen (BE); Gregory Van Barel, Berchem-Antwerpen (BE)

(21) Appl. No.: 17/267,902

(22) PCT Filed: Aug. 19, 2019
(86) PCT No.: PCT/EP2019/025273

§ 371 Date: Feb. 11, 2021

(30) Foreign Application Priority Data
Aug. 20, 2018 (BE) BE2018/5577

(51) Int. Cl. B24C 3/06; B24C 5/06; B24C 7/00

(52) U.S. Cl. CPC B24C 3/06; B24C 7/0023; B24C 5/064

(57) ABSTRACT

The present invention relates to a portable and 360 degrees operational particle blast system comprising a blast device (35) for blasting particles, comprising a blasting wheel (6), the blasting wheel comprising a rotor (6A), having blades (14) for accelerating the particles to be blasted through an exit mouth (33) of the blast device, a stator (6B) with a control cage (17), the blast device further comprising a control system (10). The latter comprises a controller for controlling the speed and the flow rate of the particles to be blasted. The system further comprises a removable, pre-filled, closed recipient (7), suitable to be operationally connected to the blast device, said recipient containing the particles to be blasted, and further comprising i.a. an actuator, acting upon a movable piston (22), causing the particles to flow against the valve to the blasting wheel and means for communicating upon connection of the recipient to the blast device, to the controller of the blast device, the operational parameters, whereby the speed and the flow rate of the blasted particles are determined by the controller solely as a function of the operational parameters received from the recipient.

### FIELD OF THE INVENTION

The invention relates to a particle blast system and device, also known as a blasting machine, sandblaster or grit blaster. Such a particle blast device serves to remove a layer of material from a surface. The removal of a layer of material can be limited to a certain zone, making it possible to create a slot or opening.

### BACKGROUND OF THE INVENTION

In professional jargon abrasive treatment of surfaces by impacting particles is often referred to as sandblasting. This surface treatment technique removes existing surface layers completely or partly and results in a roughening of the impacted surface.

In order to remove a layer of material (such as paint or rust) from surfaces such as wood, metal, stone, glass or plastic, it is possible to use simple sandpaper, a portable sander, a chemical stripper, or a paint stripper. The disadvantage of these tools is that the sandpaper requires manual labor, that the portable sander is suitable for large, flat surfaces only that the chemical stripper is limited to paint and cannot be used for precision work and can cause irritation, and that the paint stripper is also limited to paint and not usable for precision work, and can cause a penetrating smell of warm paint. Alternatively, steel wool or a wire brush can also be used.

Sandblasting is a surface treatment of materials in which particles are blown or thrown against an object in order to achieve a sanding effect. The materials can be hard materials and soft materials. Examples of soft materials are plaster and brick layer. Shot blasting is advantageous over the aforementioned techniques in that it requires less manual labor than sandpaper, it is suitable for surfaces that are not flat, it does not use a chemical stripper and that it can be used for precision work. Examples of precision work are sculpted wood objects, plaster objects like molded ceiling and other objects with an irregular surface where the shape is retained after blasting.

Typical applications for grit blasting are: removing rust or paint from a surface, making surface structures on, for example, glass or bronze and creating images on stones.

There are several types of particle blast devices known.

U.S. Pat. No. 2,723,498 is an example of a grit blaster that uses a compressor and compressed air. An advantage is that it is a semi portable grit blaster. An important disadvantage is that the grit blaster, despite the portability, is still connected to a compressor. A compressor is typically not portable, so the flexibility of this type of grit blaster is still limited to a region around the compressor. A further disadvantage of this type of grit blaster is that it is expensive because a compressor is necessary. An additional disadvantage is that the compressor also requires separate operation. A further additional disadvantage is that supply pipes for the compressed air and the grit reduce the user's freedom of movement.

U.S. Pat. No. 6,059,639 is an example where grit blasting is done with the help of a throwing wheel. In particular, a throwing wheel rotates at a speed so that the grit particles are thrown against an object. In this application the throwing wheel has a fixed arrangement, and a mechanism is provided to bring and turn objects under the throwing wheel. This type of blasting device is typically used in industry. A disadvantage of this grit blaster is that the size of objects is limited. It is also not possible to blast only parts of the object. A further additional disadvantage is that an inexperienced user might use abrasive material which is not suited for the machine and therefore might block operation of the rotating components, hence creating a dangerous situation.

U.S. Pat. No. 5,514,026 is an example of a portable refillable canister for grit blasting. An advantage is that it is a portable, cheap grit blaster. Important disadvantages are the lower power and efficiency of this grit blaster. It will not work upside down (360° orientability)

U.S. Pat. No. 4,057,938A is a portable grit blasting device which has no fixtures needed for support. Important disadvantages are the pouch which contains the abrasive which must be carried on the body and the need of two hands operation of the device. This is cumbersome for the operator and might cause a dangerous situation (e.g. when standing on a ladder) due to instability.

KR101736624B1 is a grid blaster that allows control by an operator through a central control system of blast angle, position and flow. An important disadvantage for the inexperienced user is to know and understand the correct setting of the operational parameters of the blast process for a specific job.

The goal of the present invention is to provide:
A fully portable particle blast system and device that can be hold in only one hand and that is able to operate under all possible angles, hence against gravitation. The absence of any fixtures, tubes or separated container results in a safe operation at places which are difficult to reach.
A powerful particle blast system and device to grit blast surfaces.
A closed system which does not allow (re-)filling by the user, to avoid any hazardous operation.
A smart system which does not need operational input or instructions from the user or operator.

### SUMMARY OF THE INVENTION

Therefore, the present invention relates to

a portable and 360 degrees operational particle blast system comprising
a blast device (35) for blasting particles, comprising a blasting wheel (6) driven by a motor, the blasting wheel comprising a rotor (6A), having blades (14) for accelerating the particles to be blasted through an exit mouth (33) of the blast device, a stator (6B) with a control cage (17), the blasting wheel further comprising a central axial (34) opening through which the particles are fed to the blasting wheel, the blast device further comprising a control system (10), the latter comprising
   a controller for controlling the speed and the flow rate of the particles to be blasted;
   a receiver for receiving operational parameters from a recipient and transferring same to the controller;
   means for communicating operational parameters to a user;
a removable, pre-filled, closed recipient (7), suitable to be operationally connected to the blast device, said recipient containing the particles to be blasted, and further comprising
   a valve, suitable to be opened upon operational connection of the recipient to the blasting wheel;
   an actuator, acting upon a movable piston (22), causing the particles to flow against the valve to the blasting wheel;
   means for communicating upon connection of the recipient to the blast device, to the controller of the blast device, the operational parameters
whereby the speed and the flow rate of the blasted particles are determined by the controller solely as a function of the operational parameters received from the recipient.

Further preferred embodiments of the present invention are set out in the claims, in particular in the dependent claim, the content whereof is incorporated in the description by reference.

By the term “solely” as mentioned above is meant that the controller controls the function of the blast device exclusively or solely based on the input received from the tag of the recipient, namely the operational parameters. So, there is no need for the user of the blast system to further steer the system on the basis of user's needs or preferences.

By the term “pre-filled” as mentioned above is meant that the recipient is filled with particles by the manufacturer of the recipient, and upon being used up (empty) cannot be re-filled by the user again.

By the term “against the opening” as mentioned above is meant that at any time during the blasting operation, a void between the particle mass and the opening should be avoided. Tests performed by the inventors have revealed that upon occurrence of such void or differently phrased when the contact between the particles and the opening is being broken, the flow of particles to the blasting wheel is interrupted and is difficult to be restored again.

### DETAILED DESCRIPTION OF THE INVENTION

A particle blast device ejects particles with a predetermined size.

The particle blast system as described hereinafter is ‘360 degrees operational’; this means that the system can be used by an operator under any spatial orientation; so the system can be used to blast surfaces positioned underneath of positioned below the system. In that case the particles are blast downwards by the operator or user.

Alternatively, the system can be used to blast surfaces or objects positioned above the system. In that case the particles are blast upwards by the operator or user to the surface to be cleaned.

These particles are stored in a canister, which forms a controlled environment. Other names for the canister are can, capsule or recipient.

In the appended claims, the term recipient is used, being a synonym for can or capsule which are used throughout the description that follows. Furthermore, the capsule contains an opening via which the particles can move to a blasting or throwing wheel. A valve can regulate this opening. The valve is defined as an actuator, part of the control system that controls the flow of the particles. The delivery of particles can take place in controlled quantities per unit of time. The blasting or throwing wheel is designed to rotate at a speed about its axis, whereby the particles are accelerated via blades to a certain speed. The can contains the particles in a zone that has an opening. As a result, the particles are available on the throwing wheel via the opening and/or valve. The valve has the advantage that the supply of particles can be controlled. For this purpose the valve can vary the diameter of the flow through opening. The particles can be delivered to the blasting wheel in various possible ways. Examples of this are by means of gravity and by means of pressure. The throwing wheel also causes an additional suction force for the particles during its rotation. The use of a can or capsule operationally or operably connected to the blasting wheel has the advantage that the particle blast device is portable. The particles are stored in the can in a portable quantity. Furthermore, the cans are replaceable, so that the particle blast device is reusable and/or can be used with different types of particles. The can is pre-filled at the factory and avoids refill by the user, hence resulting in a safe operation. The throwing wheel is driven by a motor located in the particle blast device. The particle blast device therefore has its propulsion for the complete operation of the device, including the acceleration of the particles. Furthermore, the particles are ejected via an exit mouth or opening after the throwing wheel has accelerated them. The particle blast device can also contain several blasting wheels.

The particle blast device has a simple structure, which makes it cheap. Furthermore, the particle blast device is portable by the use of a throwing wheel for accelerating particles. This throwing wheel also ensures that the particles can be rapidly accelerated.

Preferably, the particle blast device is arranged so that the capsule contains a second zone that provide the pressure or force to press the particles towards the opening. The can is foreseen to deliver the particles to the throwing wheel. One way in which this can be done is to provide a pressurizing medium in a second zone. This pressure medium is provided to exert a force, via a piston, on the particles in the first zone, so that they have a tendency to move towards the blasting wheel. The use of a pressure medium is advantageous because the particles can move against gravity. For example, the pressure medium may be a higher pressure in the second zone of the canister so that the particles are movable towards the opening of the capsule. Another way is to use a pushing spring element in the second zone, which presses in a similar manner on the first zone, so that the particles are movable towards the opening of the capsule. Another way is to use a pulling spring element that pulls the piston and therefore also presses the particles in the first zone.

Tests have shown that the use of a can with a pressure medium in a second zone has the advantage that the particle blast device can be used in any possible angle and/or orientation. The capsule makes it possible to do this because the particles are stored in a zone that has an opening, and because the second zone presses the particles in this zone to the opening. As a result, it is possible to keep the particle blast device upside down relative to the ground without appreciably hindering the operation.

Preferably, the particle blast device is arranged so that the capsule is removably connected to the particle blast device. By making the capsule removably connected, the capsule can be replaced by another capsule. This has the advantage that the capsules can be replaced when necessary. This is the case, for example, when the capsule is empty, or a different type of particle is desired.

Preferably, the particle blast device is arranged so that the blasting wheel can be replaced. In a portable device, the throwing wheel is preferably as light as possible. The low weight of the wheel increases ergonomics and safety, especially when the wheel is accelerated to high speeds. The low weight makes the throwing wheel potentially more sensitive to wear. In any case, regardless of its weight, the blasting wheel is subject to many forces and to abrasive action of particles by accelerating the particles during the operation of the particle blast device. Making the throwing wheel replaceable ensures that the particle blast device does not become completely unusable when the wheel is worn. Alternatively, different blasting wheels can be provided for accelerating different types of particles and/or to eject particles at different speeds. It is preferably possible to replace other parts of the particle blast device. To further avoid the negative impact of wear, the blast system is arranged for monitoring the usage of the blasting wheel and to stop the motor and inform the user when usage exceeds a predetermined limit. Preferably such a safety system consists of a rubber enclosed wire loop. The loop is installed in a predetermined location in the blasting device. Once this location wears down past a point, the wire becomes frayed and/or cut, breaking the circuit which the controller will detect.

The particle blast system is preferably arranged so that it comprises a controller, provided for operating the particle blast device on the basis of the input of operational parameters. These operational parameters can be optimized so that the particle blast device works better when blasting different types of material. For example, the operational parameters for blasting soft materials are preferably different from the operational parameters for blasting hard materials. Examples of operational parameters are particle velocity, flow rate, distance to surface, angle, speed and valve position.

The particle blast device is preferably arranged so that the predetermined speed is controllable. A controllable speed of the ejected particles results in the particle blast device having a better effect in different conditions. For example, it is necessary to use different speeds in different circumstances. Conditions such as the type of material to be blasted, the humidity of the material to be blasted, the depth to be sanded, type and/or size of the particles, . . . require that a different velocity of the particles is desired.

Preferably, the particle blast device is adapted to change the angle of the ejected particles. For this purpose, for example, an exit mouth may be provided on the particle blast device. By controlling the ejection angle, the density can be adjusted at a constant flow rate. This increases usability and application range.

The particle blast device is preferably arranged so that the predetermined flow rate is controllable. An analogous reasoning can be made here. The adjustable flow rate means that the particle blast device has a better effect in different conditions. The flow rate can be controlled by the opening surface of the valve. The controller can control the opening of the valve manually or automatically.

Preferably, the determined size of the particles is at least 1 μm on average and maximum 5000 μm on average. Tests have shown that these sizes are the desired sizes for blasting different surfaces. The different sizes are supplied in different cans. This choice of sizes has the advantage that the optimum particle size and/or particle type can be used. In addition to size of the particles, irregularity of the shape and mechanical properties, such as hardness and breaking resistance, also affect the abrasion properties.

Preferably, the particle blast device is portable and has a total mass, including filled capsule, of a maximum of 25 kg, further preferably a maximum of 15 kg, most preferably 7 kg. It is an advantage that the particle blast device is portable. As a result, the particle blast device can clean at locations which are hard to reach. An example of this is grit blasting on a ladder, where the particle blast device can be operated with one hand. Another example is the use in small spaces such as under the hood of a car or under a car, where the particle blast device can be aimed at the hard-to-reach spot. Yet another example is the blasting of rust spots on wind turbine mills, fencing and corners and edges of stair treads. The particle blast device is manageable in its completeness by a user under any possible orientation. Preferably, the motor is an electric motor. By using an electric motor, the particle blast device can be used when the electricity grid is connected. Alternatively, a battery can be used as a power supply. Another advantage is that an electric motor can be built into the particle blast device. An electric motor is lighter than other types of engines, so the total weight remains lower when using an electric motor. The electric motor is preferably arranged to control the rotational speed of the blasting wheel. The adjustable speed means that the ejection speed of the particles is adjustable. This is advantageous for blasting different objects with different properties, because some materials require a higher speed for blasting than other materials.

The ejected particles are preferably orientable relative to the particle blast device. An exit mouth can influence the direction of the blasted particles. This has the advantage that the particles can be directed without repositioning the particle blast device. For example, in hard-to-reach places, where the particle blast device cannot move freely in all directions, this can be advantageous.

Preferably, the particle blast device further comprises a collecting mechanism for collecting rebounding particles to limit dust development. This minimizes the nuisance for the user. The receiving mechanism preferably comprises a protective cap for collecting the reflected particles and dust.

Preferably the particle blast device comprises a directional mechanism, for example a laser or other light source, which indicates a hotspot of the blasting on the surface to be blasted. Preferably, the particle blast device comprises a laser that forms a preferably circular pattern that fits in a rectangular crosshair when the blast is properly directed. The pattern is an oval if the blasting is not properly directed. This allows the user to adjust and to pursue the correct blasting angle. The diameter determines the distance and fits just in a square frame, for example a flat laser curtain, or four dots or scale, if the correct distance from the target is used. Every type of particle has an optimal blasting distance and angle for a particular finish. The control of the laser is preferably done via the controller, which in turn receives an input signal from the type of container. The craftsman will recognize that the projected pattern can also have another shape, which corresponds to the pattern of the ejected particles.

### DRAWINGS

The invention will now be described in more detail with reference to an illustrative embodiment shown in the drawings.

In these drawings:

FIG. 1 shows a schematic representation of an object being blasted;

FIG. 2 shows a schematic diagram of a particle blast device;

FIG. 3A shows a schematic perspective view of a rotor of a blasting wheel;

FIG. 3B shows a schematic perspective view of a stator of a blasting wheel;

FIG. 4 shows a schematic cross-section of different embodiments of a capsule;

FIG. 5A shows a schematic perspective view of a particle blast device according to an embodiment of the invention; and

FIG. 5B shows a schematic perspective view of a particle blast device according to a second embodiment of the invention.

FIG. 6 shows an alternative embodiment of a blast wheel device.

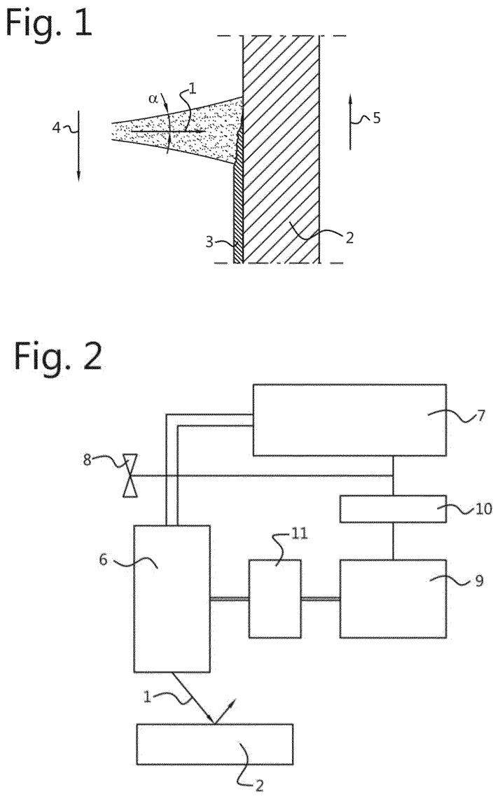

FIG. 1 shows a schematic representation of an object that is being blasted. The blasting is done by a ray 1, consisting of particles. These particles move in a direction towards an object 2. The object comprises a top layer 3, which can be removed by the impacting particles 1. The particles hit the object at a certain speed, as a result of which the top layer 3 of the object becomes eroded and therefore, at least partially, removed. The top layer 3 can be the same material as the object 2 or another material than the object 2.

The ray of blasted particles 1 can contain different types of particles. These particles can be metallic or non-metallic. Typical examples of non-metallic grit are ground coconut shell, dry ice, carborundum (silicon carbide), sodium bicarbonate (soda), olivine or glass beads. Preferably, the ray of blasted particles 1 contains only one kind of particles. Further preferably they are of substantially the same size.

The size and/or mechanical properties of the particles used have an influence on the abrasive process. For example, larger, harder and/or rougher particles are typically used for removing a difficult to remove top layer 3. Furthermore, smaller, softer and/or smoother particles are used for precision work. Preferably a mix of different particle sizes is used. Alternatively, the particles in the ray 1 are almost equally large. Preferably, capsule 7 is filled with one and the same type of particles. Alternatively, capsule 7 can be filled with different types of particles.

A particle size can typically be chosen between 1 μm and 5000 μm. Preferably, the average particle size is less than 2000 μm, more preferably less than 500 μm, most preferably less than 100 μm. Preferably, the average particle size is greater than 10 μm, more preferably greater than 20 μm, most preferably greater than 50 μm. Virtually the same size is defined as: at least 99% of the particles in capsule 7 exhibit a deviation of less than 50% of the average particle size with respect to the average particle size, furthermore preferably less than 25%, most preferably less than 20%.

Preferably, the particles are not conductive. Non-conductive particles can be used for blasting objects where there is a risk of explosion or electric shock. Examples where there is a risk of explosion are the blasting of a mounted petrol tank or the blasting of parts of an aircraft. Examples where there is a risk of shock are the blasting of electrical appliances, cables and contacts. By using non-conducting particles, the particle blast device will be able to operate spark-free and the particles will not generate any sparks in the space and/or on the surface to be irradiated.

The ray of blasted particles 1 has some characteristics that have an effect on the energy of the particles. The energy of the particles will be transferred to the object 2 upon impact of the particles on the top layer 3. This collision of the particles having a certain energy creates an impact. The impact causes removal of the top layer 3. In other words, due to the impact of the particles, high material stresses will be created in the top layer which, if sufficiently high, will exceed the fracture properties of the material of the top layer. The ray of blasted particles 1 has a flow rate, acceleration angle β, also called exit angle, an angle of incidence and a speed. The flow is determined by the number of particles per second that is emitted. A higher flow rate results in more particles colliding with the top layer 3 of the object 2. Because more particles collide with the top layer 3 of the object 2, the number of particles that exert impact per unit time and area on the top layer 3 is higher. Furthermore, due to the increased number of particles, the total energy of the ray 1 is higher. This has the advantage that the top layer 3 can be removed more quickly. The acceleration angle β has a direct relationship with the density of the blasting. If the acceleration angle β is greater, then the density of the particles in the blasting is smaller when keeping other parameters the same. The acceleration angle β can be chosen in function of the surface of the object 2 being irradiated. A larger acceleration angle β covers a larger surface area of the object 2. A smaller angle of acceleration 13 covers a smaller area of the object 2. This is advantageous, because a choice can be made between more precise or less precise blasting. The angle of acceleration can also be called a blasting angle. The acceleration angle can be defined as the diverging angle of the radius in the plane of the rotating wheel. The acceleration angle can also be called spreading angle or ejection angle.

The angle of incidence of the particles on the surface of the object 2 also has an influence on the energy that is transferred. A perpendicular angle ensures maximum energy transfer. A smaller angle ensures a lower energy transfer. The angle of incidence has an optimal angle, depending on the type of particles, the type of top layer 3 and other factors. However, angle of incidence is often not adjustable. The angle of incidence is furthermore dependent on the acceleration angle and is different for different places of incidence of the particles.

The velocity of the particle in the ray of blasted particles 1 determines the energy of the particle together with the particle size and the particle type. A higher velocity of the particles increases the friction on and/or erosion of the top layer 3 of the object 2. As a result, a top layer 3 which is more difficult to remove can be blasted.

Blasting can serve different purposes, such as removing a complete top layer 3, or blasting shapes into objects 2. Typical examples of removing a complete top layer 3 are removing paint and rust from an object 2. A typical example of blasting shapes in objects 2 is printing text in stone or using templates with decorative figures or a watermark in glass.

The particle blasting 1 and the object 2 are movable with respect to each other. The ray of blasted particles 1 and/or the object can be moved. The particle blast stream 1 can perform a movement 4 relative to the object, and/or the object 2 can perform a movement relative to the particle blasting. The particle blasting 1 and the object 2 are also movable simultaneously. The movability of the blast stream 1 and/or the object 2 makes the ray of blaster particles 1 controllable and/or orientable, so that it radiates a desired zone of the top layer 3.

FIG. 2 shows a schematic diagram of the particle blast device. The particle blast device comprises a blasting or throwing wheel 6. The throwing wheel 6 is operatively connected to a capsule 7 for supplying particles stored in the capsule 7 to the throwing wheel 6. The throwing wheel 6 is provided with particles to speed up by turning around at a speed. Furthermore, the throwing wheel 6 is provided for ejecting the accelerated particles. As a result, a ray of blasted particles 1 is formed. The particle blast device is orientable so that the ray 1 can be controlled in the direction of an object 2.

The capsule 7 contains the particles. The can 7 is provided to supply the particles to the throwing wheel 6. Preferably the capsule 7 is replaceably connected to the throwing wheel 6, directly or indirectly. Making the capsule 7 replaceable ensures that the particle blast device can be used with different capsules 7. As a result, the total weight of the particle blast device is kept lower, since the capsule 7 can be regularly replaced and thus the weight of the capsules can be limited. Furthermore, it is also possible to provide a different type of particles to the particle blast device by replacing capsule 7.

The connection between the throwing wheel 6 and the capsule 7 is controllable by means of a valve 8. For example, the valve 8 is an iris valve. The valve 8 is provided to control the passage of the particles to the throwing wheel 6. Particles provided on the throwing wheel 6 are accelerated and ejected. By reducing the number of particles that are supplied to the throwing wheel 6, the 9 flow is reduced. The valve 8 is provided to regulate the flow. Preferably, the valve is provided in the capsule and the valve is mechanically at least partially opened when mounting, e.g. screwing, the capsule on the apparatus.

Further, the particle blast device 35 comprises a motor 9 which is connected to the throwing wheel 6 for supplying power to the throwing wheel 6. The motor 9 can be connected to 5 the throwing wheel 6 via a transmission 11. The motor can also be connected directly to the throwing wheel. The particle blast device 35 comprises a controller 10. The controller 10 communicates with the capsule 7, the valve 8, the laser (not shown) and the motor 9 for receiving information and/or controlling the aforementioned elements. In operation, the throwing wheel 6 is driven at a predetermined speed. This speed is preferably higher than 2500 revolutions per 10 minute (rpm), more preferably higher than 3500 revolutions per minute. The speed is preferably lower than 40,000 revolutions per minute and more preferably lower than 30,000 revolutions per minute.

The operation of the particle blast device 35 can be summarized as follows. The particle blast device is provided to radiate a top layer 3 of an object with accelerated particles. These accelerated particles form a ray 1. The particles are typically stored at the start in a capsule 7. Valve 8 controls the flow of the particles from the capsule 7 to the throwing wheel 6. The particles are entrained in a rotation by the throwing wheel 6, which are then accelerated and ejected into the stream of particles 1. The throwing wheel 6 rotates, causing the particles to be accelerated by a blade (not shown in this figure). Turning the throwing wheel 6 is obtained by providing the throwing wheel 6 with a drive by the motor 9. The particle blast device 35 is directed towards an object 2 where the top layer 3 is removed by the impacting particles. The characteristics of the particle blasting 1 are controlled by the controller 10. The controller 10 preferably controls the valve 8, the motor 9 and the laser. The motor can also drive the throwing wheel via a transmission 11.

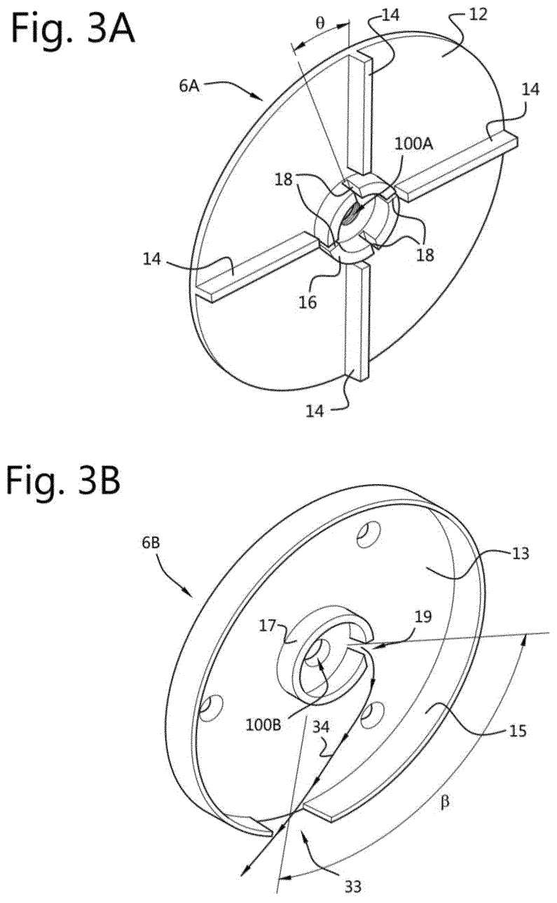

FIGS. 3A and 3B show respectively a rotor 6A and a stator 6B, which are parts of the throwing wheel 6. The rotor 6A is arranged to rotate in stator 6B to mechanically accelerate particles.

FIG. 3A shows a schematic perspective view of the rotor 6A of the throwing wheel 6. Rotor 6A comprises a rotor body 12. Preferably the rotor body 12 is made of a light material, more preferably a light metal, in which a connecting opening 100A is provided centrally. Preferably, the connection opening 100A serves for connecting the motor 9 to the rotor 6A. If the motor 9 is connected to a transmission 11, the transmission is preferably connected to the rotor 6A via the connection opening 100A. Alternatively to a connecting opening, another connecting element can also be provided with which the rotor body 12 can be connected to the motor or transmission. Around the connection opening 100A an accelerator 16 is provided, which is designed as a ring which rises from the rotor body 12. When rotor 6A rotates, particles which are brought into the center of the accelerator 16 end up in grooves 18. Due to the rotation of the rotor body 12, the particles in the grooves 18 will be rotated along with the rotor body. As a result of the rotation of the particles, the particles will also experience a centrifugal force and thus be forced in the radial direction. As a result, the particles experience an acceleration directed from the connection opening 100A to the outer circumference of rotor body 12. In accelerator 16 one or more slots 18 are provided. The person skilled in the art will understand that the number of slots 18 and the dimensions of the slots may differ per embodiment. The accelerator 16 provides a first acceleration of the particles.

These multiple slots 18 are arranged so that the particles escape from the accelerator when rotor 6A rotates. The particles are accelerated by the centrifugal action of the accelerator 16 and escape radially from the accelerator 16 through the escape opening 19 from the control cage 17, while the particles follow the rotational movement of the rotor 6A during the first acceleration.

Outside accelerator 16, vanes or blades 14 are provided. The vanes 14 extend substantially radially from accelerator 16 in the direction of the outer circumference of rotor body 12. Preferably there are as many vanes 14 as there are slots 18. FIG. 3 shows an embodiment with four blades. The person skilled in the art will understand that a rotor can also be formed with fewer than four or with more than four blades.

The vanes 14 extend at an angle θ to a direction of a nearby slot 18. Accelerated particles can thus be carried along by the vane 14, which escape from the adjacent slot 18 as they are accelerated by the accelerator 16. The accelerated particles further accelerate along the side of the blade 14. When rotor 10A rotates, the blades 14 further accelerate the entrained particles until they can be ejected at the outer circumference of rotor body 12. Preferably, the angle θ is smaller than 45°, further preferably smaller than 30°, most preferably smaller than 15°. The shape, dimensions and position of the blade can be optimized on the basis of tests and simulations.

When particles escape from the accelerator 16, the particles are entrained by blade 14. The blade 14 provides a further acceleration, as a result of which particles are accelerated from the escape opening 19 by the blades 14 along the path 34 to an ejection opening 33. This accelerating path 34 extends over an angle β. This angle β is preferably adjustable depending on the rotational speed of the blasting wheel 6, such that particles move mainly directly from the escape opening 19 to the discharge opening 33. Preferably, discharge opening 33 and/or the escape opening is movable, so that the angle over which the acceleration path 34 extends is adjustable.

FIG. 3B shows a schematic perspective view of the stator 6B of the throwing wheel 6. Stator 6B comprises a stator body 13 with an upright edge 15 at its outer circumference, in which discharge 11 opening 33 is provided. The raised edge is provided to form a closed assembly at the combination of stator 6B with rotor 6A. The feed opening 100B is arranged to allow the passage and delivery of particles in the accelerator 16. A control cage 17 is provided around the supply opening 100B. The control cage 17 is designed as a ring that rises from stator body 13. Control cage 17 is sized to fit in shape around accelerator 16 of rotor 6A. In control cage 17 is an escape opening 19 provided, along which particles accelerated in accelerator 16 escape through each of the slots 18. The escaped particles are carried along by a blade 14 which can rotate between control cage 17 and upright edge 15. The particles are thus further accelerated until it is ejected via discharge opening 33. Preferably, the ring of the control cage 17 at the position of the escape opening 19 is chamfered at one end. This allows the particles to escape effectively and unhindered.

Preferably, a wear resistant coating, cover plates or an abrasion resistant material is provided on at least a portion of rotor 6A and stator 6B to protect against wear by the particles. In particular, the surfaces which in normal use come into direct contact with the particles are provided with the wear-resistant coating.

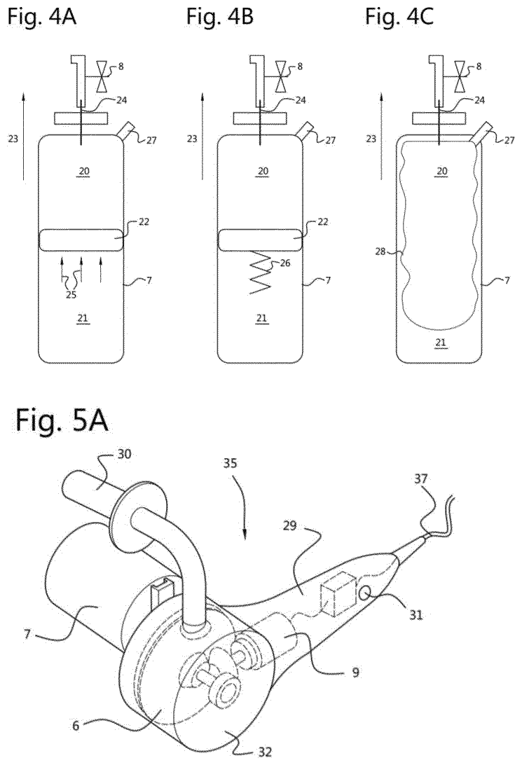

FIG. 4 shows a cross-section of different embodiments of the capsule 7. The capsule 7 contains a first zone 20 which is filled with the particles. The capsule 7 further comprises a second zone 21, a piston 22 movable in a direction of movement and an opening 24. Preferably, the direction of movement is a direction towards the particles.

The opening 24 is provided in the first zone 20. The opening 24 lies in front of the valve 8 and the throwing wheel 6. The opening 24 can be formed by the hollow interior of a pipe, tube or duct. The valve 8 may be provided in the capsule 7, at the inlet opening 100b, and/or in a connecting piece (not shown) between the capsule 7 and the throwing wheel 6. Examples of a connecting piece are a hose or a tube (not shown).

The second zone 21 preferably comprises a pressure medium 25. In a first embodiment, shown in FIG. 4A, the pressure medium 25 is a higher air pressure, that is to say an air pressure which is at least higher, preferably considerably higher than the ambient air pressure. In a second embodiment, shown in FIG. 4b, pressure medium 25 is a spring element 26. Alternatively, as shown in FIG. 4c, the pressure medium 25 and the piston 22 can be replaced by an elastic bag 28. The pressure medium 25 is provided to exert a pressure on the first zone via a separating element or piston 22. The force exerted on the separating element that provides pressure on the particles in the first zone is in the direction of the opening 24, as a result of which the particles are movable. When mechanical force is used, the piston preferably has a small opening that connect first and second zone to equilibrate the pressure in both zones. Particles are preferably pushed against the opening at all times and under each orientation of the capsule, so that the blasting process can proceed uninterruptedly. A direction of movement of the particles 23 is in the direction of the opening 24. 12 Preferably, the capsule contains a liquefying opening 27 to liquefy the particles. The liquefying opening 27 provides air to the first zone 20 of the capsule 7. Preferably, this air is provided in the vicinity of the opening. The provision of air ensures that the solid particles start to behave like a liquid, so that the delivery of the particles to the throwing wheel 6 is simplified. The air can be supplied actively, by blowing air to the liquefying opening 27, or provided passively by creating an opening to the ambient air. Preferably, the liquefying aperture 27 is formed by a perforating device (not shown) which punches through the housing of the capsule when the capsule 7 is coupled to the particle blast device. Alternatively, the liquefying opening 27 is fixedly provided on the capsule 7. A fixed liquefying opening 27 preferably has a stop so that it can be closed during storage and transport of the capsule, and can be opened when using the capsule. Alternatively, an additional valve or system can be attached to the capsule 7. This extra valve or system can draw air into the capsule. The additional valve or system is adjustable in the amount of air drawn in, preferably in function of the outgoing volume.

The movement of the particles can be regulated via the valve 8 or via the pressurizing element 25. If the valve 8 is closed, there will be no propelling particles in the direction of the movement of the particles 23. If the valve is fully opened, the valve 8 will exert a minimum resistance against the movement of the particles moving towards it. Due to the pressure in the first zone, the pressurizing element 25 is able to push forward the particles in the direction of the moving particles 23. The particles move via the opening 24 through the valve towards the throwing wheel 6. If the pressurizing element 25 exerts more pressure, the stream of particles might increase with the same amount of valve 8. The principle might be compared to electric current, where the pressure is analogous to the voltage. The valve 8 is a resistance element. The flow of particles is analogous to the electric current. The practitioner will understand how the different elements can be set and/or configured and/or can be chosen for a desired operation. Preferably, the particles in the capsule are permanently pushed against the opening 24. Preferably the capsule 7 closes itself automatically.

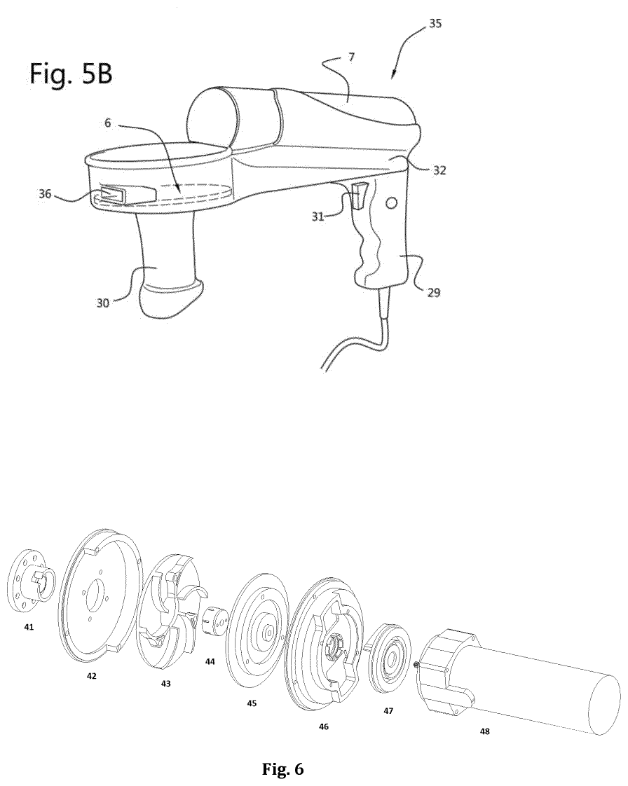

FIG. 5 shows a perspective view of two configurations of a particle blast device. FIG. 5A shows capsule 7, connected with the throwing wheel 6 that delivers the particles. The throwing wheel 6 is powered by an engine 9. Preferably at least the throwing wheel 6 and the engine 9 are protected by a frame 32.

Preferably the frame 32 consist of a nozzle or exit mouth 36 which is connected with the discharge opening. The nozzle 36 serves to orient the particle stream 1 relative with respect to the particle blast device. The spray nozzle 36 makes the acceleration α angle adjustable. Preferably the spray nozzle 36 is adjustable. The nozzle 36 can be adjusted manually, mechanically or can be adjusted by the controller 11. This makes the acceleration angle α adjustable by the controller 11. Alternatively the spray nozzle 36 is removable so that a correct spray nozzle can be mounted in function of the desired acceleration angle.

Preferably the particle blast device is equipped with at least a first handle 29 for holding the particle blast device. Furthermore, in a preferred embodiment, the particle blast device is equipped with a second handle 30.

Preferably the engine is directly linked with the throwing wheel 6. Alternatively this is done using a transmission (not shown here) linking the throwing wheel 6 and the engine 9. Preferably the engine 9 is an electric motor. By using an electric motor the particle blast device can be powered via the electricity grid 37. Alternatively it can make use of a battery (not shown) as a power supply. Another advantage is that an electric motor can be easily integrated into the particle throwing device. An electric motor is lighter than other types of engines. This limits the total weight the particle throwing device. The electric motor is preferably arranged to regulate the rotational speed of the throwing wheel. The adjustable speed means that the discharge rate of the particles is adjustable. This is beneficial for blasting of different objects with different characteristics because some materials require a higher particle speed than other materials. Alternatively the rotating speed is regulated by a transmission 11.

Preferably a control element is provided for setting the rotational speed by the user. The rotation speed is regulated by a controller 31. The control element 31 sets the rotational speed via the controller 10. The controller 10 serves to regulate the operational parameters of the particle blast device. Preferably the controller 10 regulates the opening and closing of the valve 8. The opening and closing of the valve 8 serves for controlling the flow rate of abrasive supplied to the throwing wheel 6. Preferably the controller 10 regulates the rotating speed of the object wheel 6. The throwing wheel 6 is powered by motor 9. The rotational speed of the motor 9 can be controlled by the controller or regulated by a transmission 11. The rotational speed of the wheel 6 controls the speed of the ejected particles. Preferably the controller 10 controls the pressure supplied by the pressurising device 25 via the piston 22 at the first zone. At a certain not closed position of the valve 8, a higher pressure results in a higher flow rate. The direction of movement of the particles 23 flows towards the direction of the opening 24, so they can be delivered to the throwing wheel 6.

The capsule 7 can be removably connected with the particle throwing device. Preferably, the capsule 7 is recognized by the particle blast device by means of near field communication NFC or a radio frequency identification system RFID. This has the advantage that the type of capsule 7 can be recognized. Preferably, the capsule 7 is recognized automatically, to enable the controller 10 to set the rotational speed of the engine 9 in order to achieve an optimal 14 performance of the particle blast device with the particles stored in the connected capsule 7. The particle blast device with attached capsule 7 is immediately operational when turning on the engine 9.

Preferably the particle throwing device and the capsule 7 are only usable in combination with each other, and they are unusable without each other. The particle blast device is not usable without the capsule 7 according to a configuration. The capsule 7 is not usable without the particle blast device according to a configuration. Preferably it is possible to provide a large capsule 7 that can rest on the shoulder or another body part of the professional user. A large capsule has a volume which is larger than 0.5 liters, preferably larger than 1.0 liter, more preferably larger than 2.0 liters, and, for example, a volume of approximately liters. The large capsule 7 ensures a longer operating time of one and the same capsule 7. Preferably the particle blast device works together with a vacuum cleaning device (not shown). The vacuum cleaning device serves to remove the particles after they collided with the target 2. This has the advantage that the user and the environment are less exposed to dust and particles. Optionally the particles are reusable. After purification of the removed target material, the particles can be used to fill an empty capsule 7. A second advantage is that less waste remains after the blasting process. Preferably the particle blast machine comprises a laser or other targeting device (not shown), so that the location to be blasted becomes more visible, and thereby achieving a better control of the orientation of the blasting process. Preferred properties of the laser are described above. Preferably the particle blasting machine comprises one or more removable protective screens (not shown) to protect the user against from rebounding particles or rebounding blasted material from the top layer 3. The particle blast device can also be used in an alternate embodiment for blasting of the skin of a human body. Some possibilities are the abrasive treatment of dead skin cells, teeth, bones, . . . .

An alternative embodiment of a blast wheel device, including rotor and stator is shown in FIG. 6.

In this figure, the various parts are the following:
41—Control cage
42—Stator cover or Housing
43—Curved blades
44—Accelerator
45—Rotor base
46—Stator base
47—Original part from the angle ginder, helps to lock the bearing and guide the cooling air out (the protruding part is cut off to fit under part 46)
48—The original angle grinder without the bevel gear transmission

In summary the embodiment of this invention is not only portable, but also a more practical, more mobile, more user-friendly and safer embodiment for the removal of a layer of material of a surface.

Based on the description above the professionals will understand that the invention can be performed in different ways and on the basis of different principles. The invention is not limited to the above-described embodiments. The above-described embodiments, as well as the figures are merely illustrative and serve only to increase the understanding of the invention. The invention will therefore not be limited to the embodiments set forth herein, but is defined in the claims.

1. A portable and 360 degrees operational particle blast system comprising:
a blast device for blasting particles, comprising a blasting wheel driven by a motor, the blasting wheel comprising a rotor, having blades for accelerating the particles to be blasted through an exit mouth of the blast device, a stator with a control cage, the blasting wheel further comprising a central axial opening through which the particles are fed to the blasting wheel, the blast device further comprising a control system, the latter comprising
a controller for controlling the speed and the flow rate of the particles to be blasted;
a receiver for receiving operational parameters from a recipient and transferring same to the controller;
means for communicating the operational parameters to a user;
a removable, pre-filled, closed recipient, suitable to be operationally connected to the blast device, said recipient containing the particles to be blasted, and further comprising
a valve, suitable to be opened upon operational connection of the recipient to the blasting wheel;
an actuator, acting upon a movable piston, causing the particles to flow against the valve to the blasting wheel;
means for communicating upon connection of the recipient to the blast device, to the controller of the blast device, the operational parameters, whereby the speed and the flow rate of the blasted particles are determined by the controller solely as a function of the operational parameters received from the recipient.

2. The blast system according to claim 1, wherein the operational parameters comprise one or more of the following:
size, type, hardness or composition of the particles, velocity of the blasting wheel, opening stand of the valve, type of surface to be blasted, recommended distance and orientation between the blast device and the surface to be blasted.

3. The blast system according to claim 1, whereby the means of the recipient for communicating the operational parameters to the controller of the blast device comprises a wired or wireless communication means, preferably an RFID or NFC tag, more preferably a secured or encrypted RFID or NFC tag.

4. The blast system according to claim 1, whereby the valve of the recipient is configured such that upon connection to the blast device, the valve automatically opens.

5. The blast system according to claim 1, whereby the blast device comprises a removable blasting wheel.

6. The blast system according to claim 5, the blast device further comprising means for monitoring the usage of the blasting wheel, and according to a preferred mode, such means block the function of the blast device when the usage exceeds a predetermined limit.

7. The blast system according to claim 1, whereby the movable piston is positioned in-between two zones of the recipient,
a first zone being operationally connected to the blast device and comprising the particles and;
a second zone comprising the actuator, whereby the actuator comprises a pressurizing member, causing the particles in the first zone of the recipient to flow against the valve to the blasting wheel, the actuator preferably being selected from the following list: a higher air pressure, a mechanical spring element that pushes the piston, an elastic bag.

8. The blast system according to claim 1, wherein the actuator is positioned in the zone of the recipient containing the particles, the actuator being a mechanical spring that pulls the movable piston, causing the particles to flow against the valve to the blasting wheel.

9. The blast system according to claim 1, whereby the recipient further comprising an air gap for fluidization of the particles.

10. The blast system according to claim 1, whereby the piston of the recipient further comprising an air gap enabling the pressure difference between both zones of the recipient to stabilize.

11. The blast system according to claim 1, whereby the blast device comprising means, preferably a laser and/or a display means for informing the user with respect to the recommended direction and distance between the exit mouth of the blast device and the surface to be blasted.

12. The blast system according to claim 1, whereby preceding claims, whereby the velocity of the blasting wheel is limited to 10,000 rpm.

13. The blast system according to claim 1, having a total weight, including the filled recipient, not exceeding 25 kg, preferably not exceeding 15 kg, more preferably not exceeding 7 kg.

14. The blast system according to claim 1, wherein the blast device comprises a valve for regulating the flow of particles to the blasting wheel.

15. The blast system according to claim 1, whereby the control cage is a non-rotating part of the stator, and whereby the rotor comprises a central, axial accelerator part fitting inside such stator, suitable for rendering a first acceleration to the sucked-in particles.

16. A blast device for blasting particles, comprising a blasting wheel driven by a motor, the blasting wheel comprising a rotor, having blades for accelerating the particles to be blasted through an exit mouth of the blast device, a stator with a control cage, the blasting wheel further comprising a central axial opening through which the particles are fed to the blasting wheel, the blast device further comprising a control system, the latter comprising
a controller for controlling the speed and the flow rate of the particles to be blasted;
a receiver for receiving operational parameters from a recipient and transferring same to the controller;
means for communicating the operational parameters to a user; for use in a portable and 360 degrees operational particle blast system further comprising a removable, pre-filled, closed recipient, suitable to be operationally connected to the blast device, said recipient containing the particles to be blasted, and further comprising:
a valve, suitable to be opened upon operational connection of the recipient to the blasting wheel;
an actuator, acting upon a movable piston, causing the particles to flow against the valve to the blasting wheel;
means for communicating upon connection of the recipient to the blast device, to the controller of the blast device, the operational parameters, whereby the speed and the flow rate of the blasted particles are determined by the controller solely as a function of the operational parameters received from the recipient.

17. A removable, pre-filled, closed recipient, suitable to be operationally connected to a blast device, said recipient containing particles to be blasted, and further comprising
a valve, suitable to be opened upon operational connection of the recipient to a blasting wheel of the blast device;
an actuator, acting upon a movable piston, causing the particles to flow against the valve to the blasting wheel;
means for communicating upon connection of the recipient to the blast device, to a controller of the blast device, operational parameters, for use in a portable and 360 degrees operational particle blast system further comprising:
a blast device for blasting particles, comprising a blasting wheel driven by a motor, the blasting wheel comprising a rotor, having blades for accelerating the particles to be blasted through an exit mouth of the blast device, a stator with a control cage, the blasting wheel further comprising a central axial opening through which the particles are fed to the blasting wheel, the blast device further comprising a control system, the latter comprising
a controller for controlling the speed and the flow rate of the particles to be blasted;
a receiver for receiving the operational parameters from the recipient and transferring same to the controller;
means for communicating the operational parameters to a user; whereby the speed and the flow rate of the blasted particles are determined by the controller solely as a function of the operational parameters received from the recipient.
